(19) United States
(12) Patent Application Publication
(10) Pub. No.: US 2001/0021513 A1
(43) Pub. Date: Sep. 13, 2001

(54) Test device comprising a plate containing a multiplicity of wells with an associated metering device, as well as a kit which comprises these devices and use of the devices

(76) Inventors: Wouter Cornelis Puijk, Lelystad (NL); Gerard Johannes Ligtvoet, Almere (NL); Robbert Hans Meloen, Lelystad (NL)

Correspondence Address: YOUNG & THOMPSON, 745 SOUTH 23RD STREET 2ND FLOOR, ARLINGTON, VA, 22202

(21) Appl. No.: 09/754,379

(22) Filed: Jan. 5, 2001

Related U.S. Application Data
(63) Continuation-in-part of application No. 08/811,959, filed on Mar. 5, 1997, now abandoned.
Application No. 08/811,959 is a continuation of application No. 08/244,228, filed on Jul. 25, 1994, now abandoned.
Application No. 08/244,228 is a 371 of international application No. PCT/NL92/00213, filed on Nov. 20, 1992.

(30) Foreign Application Priority Data
Nov. 21, 1991 (NL) 9101953

(51) Int. Cl. G01N 33/53; G01N 33/537; G01N 33/543

(52) U.S. Cl. 435/7.92

(57) ABSTRACT

A test device comprises a plate containing a multiplicity of wells and an associated metering device suitable for simultaneously introducing equal volumes of reagent into different wells of the test device. The test device comprises wells which have a volume within the range of 0.1-20 μl. The ratio between the depth of the wells and the diameter is preferably less than 1:1. The test device according to the invention can be used for carrying out the bio- or immuno-chemical test such as a pepscan or ELISA.

The present invention relates to a test device comprising a plate containing a multiplicity of wells. The invention also relates to a metering device suitable for the simultaneous introduction of equal volumes of reagent into different wells of the test device. In addition, the invention relates to a method for carrying out a (bio- and/or immuno) chemical test using the test device and/or the metering device and the invention is aimed at a kit which comprises the test device and the metering device.

A test device comprising a plate containing a multiplicity of wells has already been known for years in the form of the so-called microtitre plate. The known microtitre plate is of the order of 12.5 cm×8.0 cm in size and comprises 96 wells. The diameter of each well is about 0.6 cm and the depth of each well is about 1.0 cm, so that each well can contain at most a 250 μl sample. The wells are separated by material barriers with a width of approximately 2.0 mm.

The known microtitre plate is used when carrying out diverse bio- and/or immunochemical tests. In tests of this type, photometric detection is frequently used. A very well-known example of such a test is the ELISA. In the case of photometric determinations, the bottom of a well must be uniformly covered with a layer of the sample to be analysed in order to obtain reliable results. Furthermore, this layer must have a thickness which is at least such that detectable absorption occurs, In practice, this generally implies that samples are used which have at least a volume of 50 μl.

Since bio- and/or immunochemical tests frequently involve large numbers of tests on samples (such as blood and sera) which have to be obtained from test persons and/or animals, there is a need to use as little sample as possible per test.

However, the sample can be diluted to only a limited degree since the component to be analysed must also be present in the well in a certain minimum concentration in order to obtain measurable absorption. This is because, according to Lambert-Beers' law, the light intensity is dependent on the concentration and the absorption coefficient of the component to be analysed and also on the distance the light has to travel through the sample to be measured. In practice, this implies the use of about 12.5 ml of reagents per microtitre plate. In such cases there is an appreciable need to seduce the amount of sample to be used.

Another frequent use of the current microtitre plate is in the synthesis of peptides. In such syntheses peptides containing different amino acid sequences can be synthesised. This can be carried out, for example, with a view to determining the location of an epitope of a protein for a specific antibody. To this end, peptides containing amino acid sequences corresponding to a fragment of the protein to be studied are synthesised separately. The synthesis can be carried out in such a way that each peptide in part contains the amino acid sequence of another peptide. It is even possible to carry out the synthesis in such a way that only one amino acid does not overlap. It is also possible to produce a series of short sections, for example hexapeptides which overlap with the exception of one amino acid. A determination is then carried out to establish with which peptide antibody binding takes place. Peptides with which antibody binding takes place contain an epitope.

In the first instance, peptide synthesis was carried out by adding the amino acid to be coupled to the well of the known microtitre plate in which the peptide had to be synthesised, then coupling the desired amino acid to the growing peptide chain, subsequently washing the well to remove any unreacted amino acid and repeating the procedure with the next amino acid.

However, with this method problems were experienced in rinsing the wells and, therefore, a method of peptide synthesis was adopted in which small polyethylene rods are used as supports for the growing peptide chains. This method is described by Geysen, H. M., Meloen, R. H. and Barteling, S. J. beschreven in Proc. Natl. Acad. Sci. USA, Vol. 81 (July 1984) pp. 3998-4002. In this article, a method is described for the simultaneous synthesis of hundreds of peptides on a solid support with adequate purity for carrying out an ELISA. Interaction of the peptides with antibodies can be detected simply, without removing the peptides from the support. Consequently, it becomes possible to determine an immunogenic epitope with a good resolution. This method is termed the PEPSCAN.

With this method, the growing peptide chains are allowed to adhere to polyethylene rods (having a diameter of 4 mm and a length of 40 mm) and the reactions required for peptide synthesis are then carried out using the ends of the support rods. To this end, the polyethylene rods are first immersed in a 6 percent solution of acrylic acid in water and subjected to γ-radiation. For the subsequent reactions, the ends of the rods are then brought into contact with a Teflon plate containing a matrix of wells corresponding to the location of the rods (the known microtitre plate). The conventional methods for peptide chemistry in the solid phase can be used here, for example for coupling N^{α}-t-butyloxycarbonyl-L-lysine methyl ester to polyethylene/polyacrylic acid via the N^{α}-amino group of the side chain. [(Erickson, B. W. and Merrifield, R. B. (1976) in The Proteins, Eds. Neurath, H & Hill, R. L. (Academic, New York), Vol. 2, pp. 255-527) and (Meienhofer, J. (1973) in Hormonal Proteins and Peptides, Ed. Li, C.H., (Academic, New York), Vol. 2, pp. 45-267)]. After removing the t-butyloxycarbonyl group, t-butyloxycarbonyl-L-alanine can be coupled, a peptide-like spacer being formed. The desired amino acids can be coupled successively and, following the final desired coupling reaction and after removal of the protecting t-butyloxycarbonyl group, the terminal amino acid can be acetylated using acetic anhydride in dimethylformamide/triethylamine. All coupling reactions carried out with N,N-dicyclohexylcarbodiimide can be carried out in dimethylformamide in the presence of N-hydroxybenzotriazole. Any protective groups in side chains of amino acids used in the peptide synthesis can also be removed. Before the synthesised peptides are examined further, for example by means of ELISA, the rods can be washed with a phosphate-buffered saline solution.

Another use of peptide synthesis takes place if one or more amino acids of a known epitope are changed in order to determine which other sequences are able to function as an epitope and/or in order to determine which amino acids are essential for the epitope action. A method of this type is described by Geysen, H. M., Meloen, R. H. and Barteling, S. J. in Proc. Natl. Acad. Sci. USA, Vol. 82 (January 1985) pp. 178-182.

As such methods frequently comprise a large number of syntheses and thus also the use of large amounts of reagents, which reagents, moreover, are frequently expensive, there is also a need, with a view to reducing costs, to use sample amounts which are as small as possible. Possibilities have therefore been sought for miniaturisation of such peptide syntheses.

A method for miniaturised peptide synthesis has recently been described in an article by Fodor, S.P.A. et al. (Science, (15 February 1991) pp. 767-773). In this method light is used to control the simultaneous synthesis of a large number of different chemical compounds. Synthesis takes place on a solid support, such as a glass plate. The support is amniated by treatment with 0.1% aminopropyltriethoxysilane in 95% ethanol. Here, a light-sensitive protective group is then introduced, said protective group disappearing following irradiation with light and giving a reactive site to which a building block, such as an amino acid, can be coupled. The pattern in which exposure to light or other forms of energy takes place (for example via a mask) determines which areas of the support are activated for chemical coupling. The entire surface is brought into contact with the building block to be coupled (said building block also being provided with a light-sensitive protective group). A coupling reaction will occur only at sites where the light in the previous step has given rise to activation. The substrate is then exposed through another mask, so that a subsequent building block can be incorporated in the desired site. The pattern of the mask and the sequence of the reagents determine the sequences of the peptides formed. A high degree of miniaturisation can be achieved in this way. For example, it is possible to synthesise 40,000 different peptides on 1 cm².

However, this method has a number of disadvantages. The removal of the protective light-sensitive group (nitroveratryloxycarbonyl is named in the article) takes place by irradiation for 20 minutes with a mercury lamp having a power of 12 mW/cm³. This will result in a very long synthesis time in the case of the synthesis of longer peptides. Furthermore, a different mask will have to be used for each addition step and a different set of masks will have to be used for each series of peptides.

Moreover, only one building block can be added in each addition step because the various peptides to be synthesised are not spatially separated. It is obvious that mixing of reagents would otherwise take place and, thus, undesired products would also form. This method is therefore very laborious, especially for the synthesis of peptides which differ not only in respect of length but also in respect of sequence.

The authors of the article themselves also touch on the problem of the reliability of the synthesis. Deletions can occur as a consequence of incomplete removal of the protective group, following irradiation with light. The net coupling percentage is 85-95%. Furthermore, when changing masks, a certain overlap between the diverse synthesis regions will take place because of light diffraction, internal reflection and scattering. Consequently, compounds will be formed in regions which are considered to be dark, as a result of which undesired insertion of. a specific amino acid can take place.

The present invention relates to a test device which solves the miniaturisation problems described above and is suitable for use for bio- and/or immunochemical tests such as ELISA and tests in which peptide syntheses are used, for example the PEPSCAN as described above.

The present invention relates to a test device which comprises a plate containing a multiplicity of wells, which is characterised in that the wells have a volume within the range of 0.1-20 μl. The dimensions of the wells will be chosen depending on the price and availability of the samples and reagents to be used. In general, wells which are as small as possible will be preferred and, therefore, the present invention preferably relates to a test device in which the wells have a volume within the range of 0.1-5 μl.

Entirely contrary to expectations, it has now been found that making the wells smaller has no adverse consequences for the efficient rinsability thereof. It has been found that the rinsing times required in order to obtain good rinsing are shortest if the ratio between the depth of the wells and the diameter thereof is less than 1:1. Therefore, a test device comprising a plate containing a multiplicity of wells, characterised in that the wells have a volume within the range of 0.1-20 μl, and that the ratio between the depth of the wells and the diameter thereof is less than 1:1, is very suitable. A test device according to the invention for which the ratio between the depth of the wells and the diameter thereof is less than 2:3 is preferred.

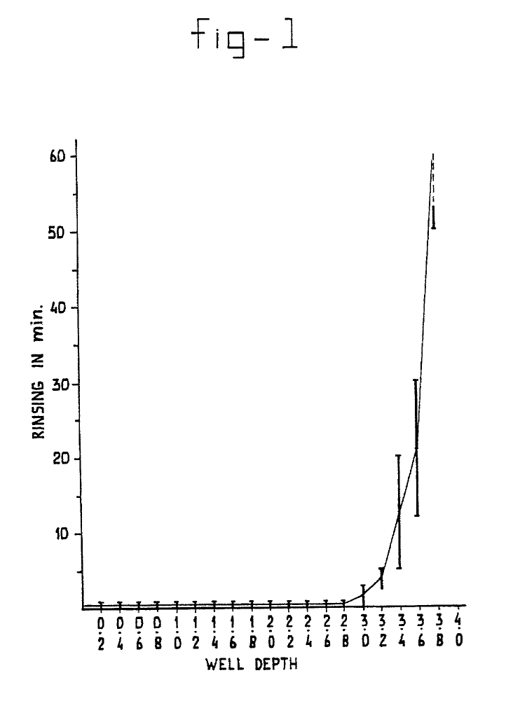

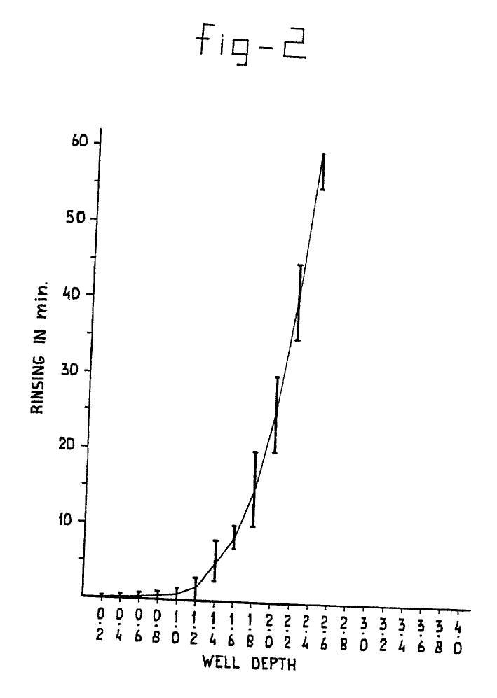

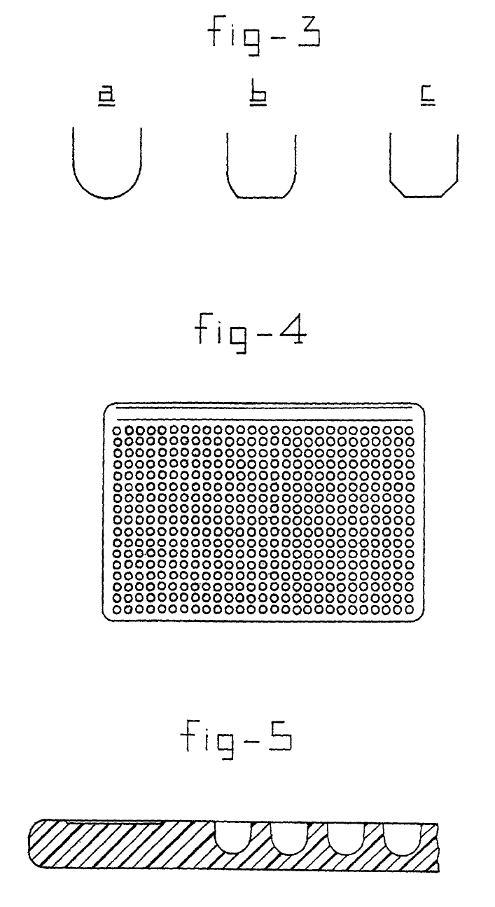

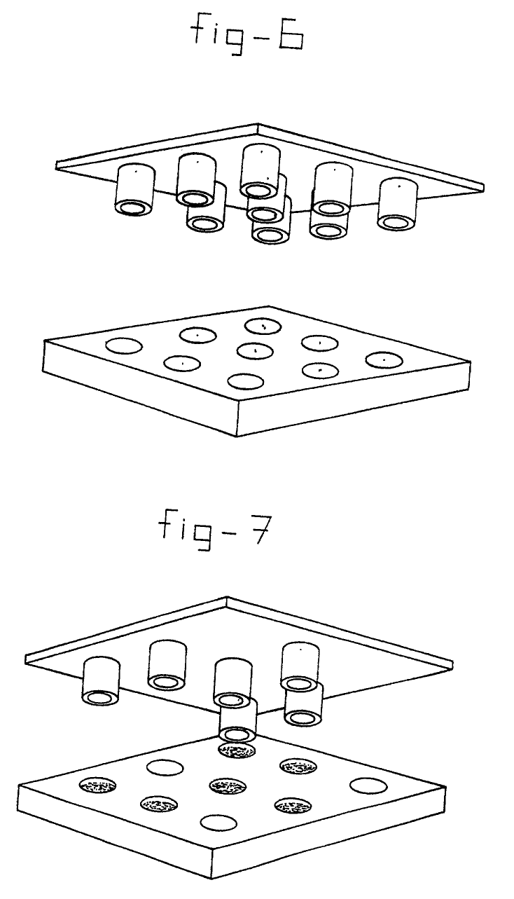

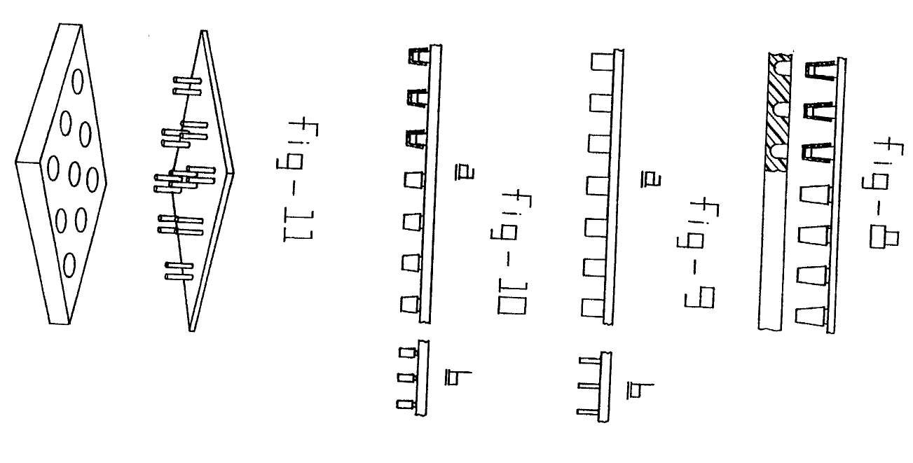

FIGS. 1 and 2 show the results of tests in which the rinsability of various test devices was investigated. The test devices had wells of equal diameter (2 mm) but of different depths. The rinsability was investigated on a shaking machine at speeds of, 47 and 40, respectively. The depth of the well in mm is plotted against the time in minutes needed to properly rinse the well.

The invention is preferably aimed at a test device in which the wells have a diameter of 1.0-4.0 mm, a diameter of 1.0-2.0 mm being preferred. The choice of the dimensions of the wells will depend on the desired specific test for which the test device is to be used. The smaller the diameter, the smaller the required volume of the sample.

In connection with the desired good rinsability, it is also preferred that the wells have a shape such that a vertical cross section of the wells is essentially U-shaped, the transition between legs and base of the U being gradual. Preferably there are no sharp angles in the well.

A number of suitable shapes of wells are shown in FIG. 3.

The U shape in which the angle between base and legs is perpendicular is preferred for photometric determinations in which measurement is carried out under and through the plate.

The test device according to the invention will preferably be a plate containing wells separated by material barriers with a width of 1.0-5.0 mm, preferably by material barriers with a width between 1.0 and 2.0 mm. The material barriers must be sufficiently wide to prevent reagents flowing over from one well to another. Specifically, the material barriers must be sufficiently wide when DMF is used as solvent, as is frequently the case in peptide synthesis. This is because DMF is known to have a high creeping capacity.

The test device according to the invention will comprise a plate containing 5-20 wells per cm², preferably 10-15 wells per cm².

Furthermore, the test device will comprise material to which peptides, proteins and other biochemical molecules, such as hormones and polysaccharides, are able to adhere. For test devices suitable for tests with peptide synthesis, such material will preferably be material to which peptides and proteins are able to adhere, such as polyethylene or poly-styrene.

Other suitable examples of materials which can be used in a test device according to the invention are polypropylene and polycarbonate.

The choice of the material for the test device will also depend on the reagents to be used in such a test device and on the detection method. In the case of photometric analysis through the bottom of the test device, for example, at least the bottom of the wells will have to be composed of transparent material. In the case of tests where DMF is used as solvent, it will not be possible to use a polystyrene test device because DMF is too aggressive.

A preferred embodiment of the test device will be provided with a means for recording information, for example a bar code or a magnetic strip. The test device can be provided with data relating to the test it is intended to carry out, or which has been carried out, such data for example relating to reagents or samples which have been used. The test device can also be provided with markings which indicate the coordinations of wells in the test device.

FIG. 4 shows a top view of an example of one embodiment of the test device according to the invention.

A cross section is shown in FIG. 5.

As already mentioned in the preamble, the present invention is also aimed at a method for carrying out (bio- and/or immuno)chemical tests, in which a test device according to the invention is used. In general, a test device according to the invention can be used in the same tests as the known microtitre plate. It is now possible to carry out existing methods using much smaller amounts of sample and reagents; so-called mini-tests are now possible. It is possible to reduce the amount of sample used by a factor of one hundred. It is now possible to use 2.5 μl instead of 250 μl samples per well. Use of the test device according to the invention now makes mass screening of population groups much more attractive because much less blood is required from the donor and much smaller quantities of reagents are required.

Another great advantage of the miniaturisation of the methods by use of the test device according to the invention lies in the fact that the existing chemistry does not have to be modified. In this context consider the great advantage, for example, in the case of automated processes, such as the PEPSCAN. The test device is particularly suitable for use in methods in which large numbers of samples have to be used. A mini-ELISA and mini-PEPSCAN in which a test device according to the invention is used are suitable examples of the method according to the present invention. The advantage of a mini-method according to the invention is that the test can be carried out with sample amounts of less than 20 μl. It is readily possible to use sample amounts of less than 5 μl in a mini-method according to the invention.

The present invention is also aimed at a metering device suitable for simultaneously introducing equal volumes of reagent into different wells in a test device according to the invention. A metering device according to the invention can be used in order to carry out as efficiently as possible immuno- and/or biochemical tests in which a test device according to the invention is used. In this context, consideration is given to optional automation of certain methods according to the invention.

If a predetermined equal amount of reagent has to be introduced simultaneously into a number of wells, it is possible to use a metering device according to the invention, said device being provided with projections having dimensions and mutual spacings such that individual projections can simultaneously be positioned in or above wells of a test device according to the invention. With a metering device of this type, all wells can simultaneously be provided with equal volumes of reagent if the position of the projections is such that this essentially corresponds to the position of the wells in the test device according to the invention.

One embodiment of the metering device and the test device is shown in FIG. 6.

If not all, but only a certain number, of wells in the test device have to be filled, a metering device can be used which has projections which simultaneously can be positioned above or in the selected wells.

In FIG. 7 the darker wells are the selected wells. The projections of the metering device are located on the metering device in such a way that they can simultaneously be positioned above or in the darker wells.

To this end, a metering device can advantageously be used in which the projections are fixed to the support or can be fixed in a pattern which corresponds to the pattern of the wells into which reagent has to be introduced.

FIG. 8 shows an example of an embodiment of the metering device in which the projections can be fixed to a support.

The metering device can comprise projections which are fixed or can be fixed to a support in a way equivalent to the bristles of a brush (FIGS. 6, 7 and 8).

A metering device in which the projections, like the teeth of a comb, are parallel to one another and are fixed (FIG. 9) or can be fixed (FIG. 10) at their tops to a support is also an embodiment of a metering device according to the invention which is very suitable. The number of projections can be less than or equal to the number of wells forming a row in the longitudinal direction of the test device. The number of projections can be less than or equal to the number of wells forming a row in the widthwise direction of the test device. The number of projections will depend on the pattern of wells of the test device into which reagent has to be introduced.

The projections of a metering device according to the invention can be integral with the support or can be detachable. The projections can be fitted on the support in such a way that the projections form a pattern which corresponds to the pattern of wells which have to be filled in the test device (see FIGS. 7).

It is also possible to use a metering device in which more than one projection can be positioned above or in a well at the same time, if it is desired to simultaneously introduce more than one reagent unit, which is present on a projection, into a well.

FIG. 11 shows a metering device in which two projections can be positioned simultaneously above or in each well.

Thus, the amount of reagent which is present on a projection can be taken as standard and metering devices can be used which have a group of projections above or in the well, depending on the ratio in which it is desired to introduce reagents into a well. A group will comprise the number of projections which corresponds to the number of desired reagent units.

In the case of a metering device according to the invention, the projections can be hollow, but they can also be solid or closed at the bottom. The latter two possibilities are to be preferred when working with very small amounts of sample and reagents, because it is then possible to work with drops of reagent.

The present invention is also aimed at a method for carrying out a (bio- and/or immuno-)chemical test using a metering device according to the invention, in which method

a) the projections of the metering device are provided with reagent, in such a way that essentially equal volumes of reagent are present on or in the individual projections of the metering device, and

b) the metering device is then positioned in or above wells of the test device according to the invention, which wells are intended to be provided with reagent, each individual projection being located in or above a well at the same time, and

c) essentially equal volumes of reagent are introduced into the individual wells of the test device, which wells it is intended to provide with reagent.

The invention also relates to a method of this type in which the projections of the metering device are simultaneously provided with reagent by immersing the projections in reagent.

The present invention is also aimed at a kit which comprises at least a test device and metering device according to the invention. Such a kit can comprise a number of metering devices in the various embodiments described above and can also comprise replaceable projections for such metering devices. For other successful applications of test-devices according to the invention, herein also called minicards resembling a credit card in size, the invention also provides a test device comprising a plate containing a multiplicity of wells wherein the wells have a volume within the range of 0.1-20 microliter, said test device grafted with hydroxymethylmethacrylate (HEMA) polymer. Use of this grafted plate allows efficient replacement netting or segment synthesis of peptides. In particular with test devices wherein the wells have a volume within the range of 0.1-5 microliter, or wherein the ratio between the depth of the wells and the diameter thereof is less than 1:1, preferably less than 2:3, and/or wherein the diameter of the wells is 1.0-4.0 mm and preferably 1.0 -2.0 mm, such ease of use is obtained, due to in the first place the rinsibility of the plates allowing various consecutive steps to be performed, and a treatment with gaseous TFA, allowing cleavage of peptide material for further synthesis in a separate, second minicard. This is in particular useful when the plate contains a high ensity of wells, such as 5-20 wells per square centimeter, preferably 10-15 wells per square centimeter.

The invention provides a method for synthesizing a library of peptides, especially of longer peptides, such as 20-mer to 50-mer peptide comprising synthesizing a first segment of said peptide in a first test device, grafted with HEMA according to the invention, synthesizing a second segment of said peptide in a second test device, preferably grafted with (poly)acrylic acid, comprising a plate containing a multiplicity of wells wherein the wells have a volume within the range of 0.1-20 microliter. The first and second segment comprise each about half of the number of amino acids as the fully synthesized peptide will comprise, e.g. a 8-9-mer can for example be coupled to a 11-12-mer, when 20-mer peptides need be obtained. Of course this numbers can vary according to convenience, in the examples a 30-mer peptide is synthesized using a first and second segment each comprising 15 amino acids, but it is to be understood that any two numbers that ad up to the desired number of amino acids of the final peptide may be used. The invention further provided subjecting said first test device to a gaseous phase comprising trifluoric acid and cleaving peptide material from the first test device, after which peptide material from said first test device is distributed to said second test device under conditions wherein peptide material from said first test device forms a bond with peptide material in said second test device. In particular, a method is provided wherein said bond comprises a thioether bond. This is further explained in detail in examples 3 and 4 hereunder.

### EXAMPLE 3

Synthesis and screening of spatially-addressed array-based combinatorial libraries Small peptide libraries composed of thousands of 12-mer peptides are synthesized in credit-card format minicards. Due to the superior signal-to-noise ratio of the minicard, in particular those grafted with (poly)acrylic acid, pepscan-elisa specific lead peptides with affinities as low as 10⁻² M have been picked up. The method is particularly useful when using peptides up to 15 to 17 amino acids long. Longer peptides require reduced synthesis rates to keep trustworthy results.

The identified lead peptides can be optimized to required affinities using the same minicards though socalled replacement analyses (method is described below in example 4) and/or through sequence analysis of all peptides.

### EXAMPLE 4

Replacement Analysis and/or Segment Synthesis of Peptides, in Particular Those Longer Than 15 Amino Acids.

In the example 30-mer peptides, made up of two 15-mer peptides are used, however, it is easily understood that somewhat smaller (i.e. from 12-mer through 20-mer up to 29-mer) to considerably larger peptides (i.e. from 31-mer through 40-mer, up-to 50-mer, or even larger constructs may be used. However, the method works best with peptide segments that are up to 15-mer long.

Using the minicards enables one to synthesize and screen thousands of peptides that have specific binding activity somewhere ≧10-2 M (see example 3 above). This makes the format very suitable for a so called replacement-net analysis or, alternatively, or in combination, for the detection of discontinuous or conformational binding sites or epitopes corresponding to a binding molecule, in particular in relation to protein-protein or biomolecule-ligand interactions. The invention allows testing for, identification, characterisation or detection of a discontinuous binding site capable of interacting with a binding molecule, said library, e.g. on the minicard, having been provided with a plurality of molecules, each molecule of said molecules comprising at least one first segment linked to a second segment, each segment having the capacity of being a potential single part of a discontinuous binding site. In an replacement-analysis each of the building blocks, i.e. amino acids, comprising a peptide is replaced by each of the other 19 amino acids, and possibly by the 19 D-amino acids, and possibly by additional non-natural amino acids such as for example cyclohexylalanine. In a replacement-net analysis the contribution of each amino acid in binding activity is investigated in full detail, i.e. amino acids that are essential for binding are identified and amino acids that improve binding activity are identified (van Amerongen et al., 1994). Normally, a replacement-analysis is used for peptides up to a length of 15 amino acids. For a 15-mer, only using L-amino acids, this amounts already up to 300 peptides including 15 controls.

The invention provides a new replacement-net analysis method using the minicards or test devices according to the invention. This has made it possible to make a replacement-analysis of peptides as long as 30-mers or even longer. Increasing the length of the peptide to say 30-mers using 20 L-amino acids, 19 D-amino acids and say 21 other non-natural amino acids would amount to a total of approximately 1800 different 30-mer peptides, a number that is easily handled within the minicard format. The 30-mer peptides are synthesized through segment coupling of two 15-mer peptides which are synthesized in two different minicards (explained in detail in methods below). The minicard format is very suitable for this approach since the microwells facilitate the segment coupling of two peptides extremely well as described below in detail.

Methods

Segment Synthesis in Minicards

card type-1

The first half of the 30-mer, i.e. a 15-mer (amino acid 1-14 with an extra carboxy-terminal cysteine, thus 15 amino acids long) is synthesized in a minicard with HEMA resin containing free amino groups (HEMA, poly-2-hydroxymethylmethacrylate-card).

Below this is described in full detail:

Peptides were synthesized at polypropylene minicards grafted with HEMA polymer. This graft polymer was made by gamma irradiation of polypropylene minicards in a 20% HEMA solution in methanol/water 80/20 at a dose of 30-50 kGy. The functionalised support can be used for the synthesis of 1 μmol of peptide/cm² after coupling of β-alanine and an acid labile Fmoc-2,4-dimethoxy-4′-(carboxymethyloxy)-benzhydrylamine (Rink) linker. The peptides were synthesised using standard Fmoc chemistry and the peptide was deprotected and cleaved from the resin by subjecting the card to a gaseous phase of trifluoroacetic acid (TFA) with scavengers, for example by holding or positioning the card above the surface of the TFA mixture (see below at Combination of the 15-mers into a 30-mer for further details).

card type-2

The second half of the 30-mer, also a 15-mer (amino acid 16-31 with an amino-terminal bromoacetamide group) is synthesized in a minicard with an acrylic acid resin polymer where the COOH-groups avia linkers are converted to free amino groups. These on the resin synthesized peptides still contain the side-chain protecting groups of the amino acid residues. Below this is described in full detail. A polypropylene or polyethylene minicard was grafted with acrylic acid. As an example: a polypropylene support in a 6% acrylic acid solution in water was irradiated using gamma radiation at a dose of 12 kGy. The grafted solid support containing carboxylic acid groups was functionalised with amino groups via coupling of t-butyloxycarbonyl-hexamethylenediamine (Boc-HMDA) using dicyclohexylcarbodiimide (DCC) and subsequent cleavage of the Boc groups using trifluoroacetic acid. Standard Fmoc peptide synthesis was used to synthesize peptides on the amino-group functionalised solid support. After cleavage of the Fmoc group of the last amino acid and washing, a bromo-group was introduced using bromoacetic acid plus DCC.

Combination of two 15-mers into a 30-mer

If a replacement-net analysis comprises for example approximately 900 peptides one requires 2 acrylic acid polymer (Ac)-cards and 2 HEMA-cards (in this example minicards contain 455 wells of 3 microliters each). The HEMA-card peptides are synthesized onto a acid-labile RINK linker that is coupled to the resin. All the HEMA-card peptides have a, preferably a carboxy-terminal but other positions are also possible, cysteine. All the Ac-card peptides have, preferably an amino-terminal, bromoacetamide group. When such two peptides are mixed (pH 7-8) the cysteine will react with the bromoacetamide group and form a stable thioether bound.

The procedure is that a HEMA-peptide in a well of the first HEMA-card is released from the well by subjecting it to the gaseous phase of a mixture of 95% trifluoric acid (TFA), 4% H₂O (mostly acting as scavenger), and 1% scavenger such as ethandithiole and/or phenol and/or thioanisole, and than transferred to.

In one embodiment, the minicard is positioned and incubated above the TFA mixture (well upright, otherwise the material gets lost from the wells after cleavage) in a closed chamber. The circumstances, such as time and temperature are not critical, the method can be executed at room temperature, and sufficiently cleaved peptide material is already obtained after 1-2 hours, and it is most often no necessary to incubate much longer than 3-4 hours. TFA and scavengers derived from the gaseous phase attack on the acid-labile RINK-linker positioned between the synthesized peptide and the solid-support of the HEMA-well. Important here is that the gaseous TFA penetrates into the HEMA-resin very efficiently thereby effectively cleaving all the peptide material. The penetration of the gaseous TFA is possible because of special properties of the HEMA resin. This is very important because it is not possible to use liquid TFA, which is a standard procedure in FMOC-peptide chemistry. In these small 3 μl wells the liquid TFA spills over very easily. This leads to spilling-over and thus loss of most of the peptide material and cross-contamination of peptides between all the wells, and is therefore not applicable.

The spacing of the wells in the minicards (10 wells/cm2, preferably fall within the specifications of a pipetting robot, such as a standard Hamilton robot. Using the Hamilton robot the peptide of well-1 of the first HEMA-card is pipetted, via a 96-wells plate, into well-1 of the first Ac-card. The cysteine of the peptide from the HEMA-card will react with the bromoacetamide group of the peptide from the Ac-card and form a stable thioether bound, thus creating e.g. a 30-mer peptide construct. Subsequent deprotection with a standard (liquid) TFA/scavenger mixture provided a minicard with long peptide constructs. Hereby the invention thus provides a test device comprising a plate containing a multiplicity of wells wherein the wells have a volume within the range of 0.1-20 microliter, said device comprising a library of peptide constructs comprising a first peptide segment linked via a non-peptide bond to a second segment. This is in particularly useful when each peptide construct essentially each comprise at least 18 amino acids, thus circumventing the inaccuracy of normally synthesized peptides of that size or larger. Best results are now obtained when said peptide constructs essentially each comprise from 25 to 35 amino acids.

References

van-Amerongen-A; Plasman-H. H.; Kuperus-D; Kremer-L; Rodriguez-Frade-J. M.; Albar-J. P.; Llopis-R; Schaaper-W. M.; Meloen-R. H. (1994). Design of peptides with improved affinities for anti-human chorionic gonadotropin monoclonal antibodies. Pept-Res. 7: 83-90

Slootstra-J. W.; Puijk-W. C.; Ligtvoet-G. J.; Langeveld-J. P.; Meloen-R. H. (1996). Structural aspects of antibody-antigen interaction revealed through small random peptide libraries. Mol-Divers. 1: 87-96.

Slootstra-J. W.; Puijk-W. C.; Ligtvoet-G. J.; Kuperus-D; Schaaper-W. M.; Meloen-R. H. (1997). Screening of a small set of random peptides: a new strategy to identify synthetic peptides that mimic epitopes. J-Mol-Recognit. 10: 217-24.

We claim:

1. A test device comprising a plate containing a multiplicity of wells wherein the wells have a volume within the range of 0.1-20 microliter, said test device being grafted with hydroxymethylmethacrylate.

2. A test device according to claim 1 wherein the wells have a volume within the range of 0.1-5 microliter.

3. A test device according to claim 1 wherein the ratio between the depth of the wells and the diameter thereof is less than 1:1.

4. A test device according to claim 3, wherein said ratio is less than 2:3.

5. A test device according to claim 1 wherein the diameter of the wells is 1.0-4.0 mm.

6. A test device according to claim 5, wherein said diameter is 1.0-2.0 mm.

7. A test device according to claim 1 wherein the plate contains 5-20 wells per square centimeter.

8. A test device according to claim 1, wherein the plate contains 10-15 wells per square centimeter.

9. A test device comprising a plate containing a multiplicity of wells wherein the wells have a volume within the range of 0.1-20 microliter, said device comprising a library of peptide constructs each comprising a first peptide segment linked via a non-peptide bond to a second segment.

10. A test device according to claim 9 wherein said peptide constructs each comprise at least 18 amino acids.

11. A test device according to claim 10 wherein said peptide constructs each comprise from 25 to 35 amino acids.

12. A method for synthesizing a library of peptides comprising
synthesizing a first segment of said peptide in a first test device according to claim 1,
synthesizing a second segment of said peptide in a second test device comprising a plate containing a multiplicity of wells wherein the wells have a volume within the range of 0.1-20 microliter,
subjecting said first test device to a gaseous phase comprising trifluoric acid and cleaving peptide material from the first test device, and
distributing peptide material from said first test device to said second test device under conditions wherein peptide material from said first test device forms a bond with peptide material in said second test device.

13. A method according to claim 12 wherein said bond comprises a thioether bond.
